(19) United States
(12) Patent Application Publication
(10) Pub. No.: US 2012/0201906 A1
(43) Pub. Date: Aug. 9, 2012

(54) NOVEL MEDICAL USES FOR NO AND NO DONOR COMPOUNDS

(76) Inventors: James D. Reynolds, Shaker Heights, OH (US); Jonathan S. Stamler, Shaker Heights, OH (US)

(21) Appl. No.: 13/394,576

(22) PCT Filed: Jul. 16, 2010
(86) PCT No.: PCT/US10/02013

§ 371 Date: Apr. 26, 2012

Related U.S. Application Data
(60) Provisional application No. 61/240,521, filed on Sep. 8, 2009.

(51) Int. Cl. A61K 31/21; C12N 5/071; A61P 43/00; A01N 1/02; A61K 33/00; A61P 11/00

(52) U.S. Cl. 424/718; 514/506; 514/509; 435/1.1; 435/374

(57) ABSTRACT

A body part is preserved using nitric oxide and/or a nitric oxide donor that does not directly release nitric oxide or a red blood cell nitrosylating agent, preferably ethyl nitrite to facilitate oxygen supply. A subject at risk for developing high altitude illness is administered a red blood nitrosylating agent in gaseous form that does not directly release nitric oxide, preferably ethyl nitrite.

### TECHNICAL FIELD

The present invention relates to novel medical uses for NO, NO donor compounds and/or mixtures thereof.

### BACKGROUND OF THE INVENTION

Nitric oxide (NO), a highly reactive and diffusible radical, plays an important role in the regulation of a wide range of physiological processes. The administration of NO, NO donor compounds, and/or mixtures thereof are effective in treating a diverse range of disorders. The present invention relates to novel medical uses for NO, NO donor compounds and/or mixtures thereof, such as facilitating organ transplants and treating high altitude sickness disorders.

### SUMMARY OF THE INVENTION

A first embodiment of the invention is a method for preserving a body part requiring a continual supply of oxygen and nutrients, comprising administration to the whole body or body part a NO donor compound, and/or mixtures thereof in an amount sufficient to maintain cellular metabolic activity and function of the body part. The body part is from a human or an animal such as a mammalian species of animal.

A second embodiment is directed to a method for treating a subject having or at risk of developing high altitude illnesses, high altitude pulmonary edema, high altitude cerebral edema and/or acute mountain sickness, comprising administration to the subject in need thereof a therapeutically effective amount of a NO donor compound, wherein said NO donor compound comprises a red blood cell nitrosylating agent in gaseous form that does not directly release NO itself. A subject means herein a human or animal such as a mammalian species of animal. The second embodiment is especially important for individuals with lung conditions going to altitude.

The term “animal” as used herein includes, for example, cats, dogs, mules, and sheep.

### BRIEF DESCRIPTION OF THE DRAWINGS

FIG. 1 illustrates the results of Example 1, which reports the effects of administering ethyl nitrite (ENO) to maintain/increase NO bioactivity after brain death.

FIG. 2 shows the results of Example 2, which reports the effects of administering ENO in maintaining in vivo organ status.

FIG. 3 shows the results of Example 12, which reports the physiological response of subjects to administration of ENO under conditions that mimic high altitude.

### DESCRIPTION OF THE INVENTION

We turn now to the first embodiment.

NO is a free radical gas that diffuses from its site of production in endothelial cells to its target, soluble guanylate cyclase (sGC), in vascular smooth muscle cells (VSMCs). In this classical NO signaling pathway, activation of sGC enhances cyclic guanosine monophosphate (cGMP) production, which in turn mediates vasodilatation. (See Coggins and Bloch, Arteriosclerosis, Thrombosis, and Vascular Biology. 2007:27 (9) p. 1877).

Previous studies have found that endogenous NO and cGMP levels fall precipitously after reperfusion of lungs that been subject to an organ donation. These studies also show that administration of NO to a lung via an adenovirus-mediated nitric oxide synthase (eNOS) gene transfer, early perfusion of the lung graft with NO donors, inhaled NO or essential cofactors for eNOS, ameliorate ischemia-reperfusion (I-R) injury, and improve graft function (See Karamsetty et al., Am. J. Respir. Cell Mol. Biol., 2002:26:1, p. 1-5).

Supplementing an organ preservation solution with a NO cGMP analog such as nitroglycerin has also been shown to improve organ graft function and improve organ recipient survival (Pinsky et al. J Thorac Cardiovasc Surg 1999:118, pg. 135-144).

However, these studies also show that the results obtained with these compounds and methods can vary depending on the type of, compound that is used and timing of the treatment. For example, it has been shown that treatment with nitroglycerin during flush/preservation, but not during reperfusion, inhibits neutrophil accumulation in the transplanted lung (see also Murakami, et al., Am. J. Respir. Crit. Care Med. 156: 454-458).

Thus, a need exists for improved compositions and methods that can facilitate organ preservation and organ transplantation prior to, during, and after the transplantation procedure.

The phrase “organ preservation” refers to procedures used for the preservation of an organ. The “organ preservation” is for a human or an animal such as a mammalian species of animal, e.g. cats, dogs, mules, sheep, and the like.

Organ transplantation is the optimal intervention for end-stage organ failure. The procedures used for procurement of the organ, the physiologic state of the donor, and the ex vivo storage time of the organ all impact whether the transplantation will be a success. Furthermore, the methods used to procure organs not only impact the procured organ but can impact the remaining organs.

The term “organ transplant” means herein the moving of an organ from one body to another or from a donor site on the patient's own body, for the purpose of replacing the recipient's damaged or failing organ with a working one from the donor site. The term includes autografts, allografts, isografts, exenografts, split transplants, and domino transplants.

An autograft is a transplant of tissue to the same person. Sometimes this is done with surplus tissue, or tissue that can regenerate, or tissues more needed elsewhere. Examples of autograft transplants include skin grafts and vein extraction for coronary artery bypass graft (CABG).

An allograft is a transplant of an organ or tissue between two genetically non-identical members of the same species. Most human tissue and organ transplants are allografts. Allografts also include isografts, wherein organs or tissues are transplanted from a donor to a genetically identical recipient, such as an identical twin.

A xenograft is a transplantation of organs or tissue from one species to another. An example is a porcine heart valve transplant, which has become increasingly common.

A split transplant is when a deceased-donor organ, such as a liver, is divided between two recipients, especially an adult and a child.

A domino transplant is a transplant that involves the removal of an organ from a donor (someone from whom an organ is taken or is to be taken) and transplantation into a patient, which in turn leads to the donation of another organ or body part to at least a second person. For example, this type of procedure is usually performed on patients with cystic fibrosis because both lungs need to be replaced and it is a technically easier operation to replace the heart and lungs at the same time. As the recipient's native heart is usually healthy, it can be transplanted into someone else needing a heart transplant. That term is also used for a special form of liver transplant in which the recipient suffers from familial amyloidotic polyneuropathy, a disease where the liver slowly produces a protein that damages other organs. This patient's liver can be transplanted into an older patient who is likely to die from other causes before a problem arises.

In one aspect of this embodiment, the inventors of the present application have discovered that the administration of NO, NO donor compounds, and/or mixtures thereof to the donor prior to transplantation increases the likelihood that the transplantation will be a success. The administration of these compounds augments the control of blood flow for oxygen delivery and vascular smooth muscle relaxation. This limits ischemic injury to the procured organ and helps to maintain the functions of the other organs of the donor.

The donor may be living, brain dead, non-heart beating, or cadaveric. A brain dead donor is typically a donor wherein lung and/or cardiac function is initially present whereas a non-heart beating, or cadaveric donor is typically a donor, wherein lung and/or cardiac function is compromised or non-existent.

Whether the donor or subject is living or dead (brain dead and/or no heart beating), the administration of NO, NO donor compounds, and/or mixtures thereof increases the number of viable organs available for transplant by utilizing a therapeutic intervention that lessens the damage sustained to both recovered and remaining organs during donation and by better preserving ex vivo organ function.

The terms “organ” and “body parts” are used interchangeably and mean herein independent parts of the body that carry out one or more functions of the body. For example, organ and body parts that can be transplanted in accordance with this embodiment include the kidney, skin, muscle, heart, lung, liver, cornea, pancreas, islets of Langerhans, intestine, stem cells, bone marrow, blood, neural tissue, and composite tissues (e.g. facial allotransplantation).

NO and NO donor compounds used herein are in dosages and routes of administration approved by the Food and Drug Administration (FDA) of the Untied States and other regulatory agencies. Otherwise dosages can be determined in bioassays by vasodilatory or anti-platelet activity.

For example, one way in which to administer nitric oxide is via a nitric oxide analyzer, which is a device subject to FDA regulation that measures the concentration of nitric oxide in respiratory gas mixtures during administration of nitric oxide. NO can be used to facilitate the transplantation of an organ selected from the group consisting of kidney, skin, liver, cornea, pancreas, islets of Langerhans, intestine, stem cells, bone marrow, blood, neural tissue, and composite tissues (e.g. facial allotransplantation).

NO is a highly reactive and readily diffusible radical. As a result, the administration of NO can generate toxic species on reaction with O₂ that induce oxidative stress and methemoglobinemia in the organs and/or body parts. (see Coggins and Bloch, Arteriosclerosis, Thrombosis, and Vascular Biology. 2007:27:1877).

A NO donor compound can be administered as a substitute for NO or in combination with NO in an effort to avoid these potentially deleterious effects. A NO donor compound is a compound that releases NO or a related redox species and more generally provides nitric oxide bioactivity, e.g., vasorelaxation or stimulation or inhibition of a receptor protein.

Compounds that contain S-nitroso groups, O-nitroso-groups, and N-nitroso groups are all known to release nitric oxide. O-nitroso compounds are compounds having one or more —O—NO groups, and are also referred to as O-nitrosylated compounds and nitrite compounds. S-nitroso compounds are compounds with one or more —S-NO groups and are also referred to as nitrosothiols and S-nitrosylated compounds. An —S-NO group is also referred to in the art as a sulfonyl nitrite, a thionitrous acid ester, an S-nitrosothiol or a thionitrite. Compounds having an =N-NO group are referred to herein as N-nitroso compounds. Other NO compounds include NONOates, nitroprusside, (FeNO compounds), nitrates, furoxans, etc. . . . Examples of these compounds can be found in U.S. Pat. Nos. 6,676,855, 6,314,956, 6,855,691, 5,824,669, 5,814,666, and 5,583,101. The entirety of each of these publications are incorporated herein by reference.

In addition, nitro compounds —Y-NO₂ are included in the embodiment (where Y is N, C, O, S or transition metal).

The NO donor compound can also be an organic nitrite or nitrate selected from the group consisting of amyl nitrate, ethyl nitrite, ethyl nitrate, isosorbide mononitrate, isosorbide dinitrate, nitroglycerin, nitrosothiols and nitroprussides.

The NO donor compounds are preferably red blood cell nitrosylating agents that do not directly release NO. These compounds are of particular interest as they influence an alternative NO signaling pathway that involves the oxidation of NO to nitrite or reactions of NO with protein thiols to form S-nitrosothiols (SNOs). SNOs can function as vasodilators.

It is believed that red blood cell nitrosylating agents that do not directly release NO can interact with hemoglobin to form S-nitrosohemoglobin (SNO-Hb), where its vasodilator potential enables selective delivery of oxygenated blood to hypoxic tissue, organs, and body parts. Because S-nitrosylation is an alternative pathway mediating many NO biological effects, treatment with red blood cell nitrosylating agents that do not directly release NO may better protect organs and body parts subject to transplantation from oxidative stress than NO (see Gatson et al., Molecular Interventions, Volume 3, Issue 5, August 2003).

In other words, these compounds do not generate pure NO upon administration, which would likely be eliminated by reactions at the hemes of hemoglobin, and likely react with O₂ and superoxide to form toxic NOx. Rather, red blood cell nitrosylating agents that do not directly release NO means herein a compound that nitrosylates the thiols of hemoglobin or that is metabolized into compounds that would nitrosylate thiols efficiently. For example, ethyl nitrite does not release NO but rather transfers its NO group to thiols to form SNO. Hence, ethyl nitrite is a nitrosylating agent that does not directly release NO. Ethyl nitrite does not react with O₂ or superoxide. One can measure the efficiency of SNO formation exhibited by compounds in vitro and in vivo (e.g. SNO-Hb production) vs. NOx formation. NO itself would be inefficient at nitrosylating thiols, is inactivated by blood hemoglobin, and forms NOx. Conversely, ethyl nitrite for example forms bioactive SNO, including SNO-hemoglobin but not NOx.

An increase in SNO-Hb is also associated with the reduction of markers of organ injury, such as creatine phosphokinase (CPK), creatinine and aspartate transaminase (AST) (e.g. Examples 1 and 2). In this regard, a red blood cell nitrosylating agent in gaseous form that does not directly release NO is preferably administered in an amount sufficient to induce in blood an increase in SNO-Hb and/or a decrease in markers of organ injury such as CPK, creatinine and/or AST.

The red blood cell nitrosylating agents that do not directly release NO are preferably gases. Examples of red blood cell nitrosylating agents in the gaseous form that do not directly release NO are ethyl nitrite, ethyl nitrate, amylnitrite, S-nitrosocysteine, S-nitrosoglutathione, or a mixture thereof. The red blood cell nitrosylating agents in the gaseous form that do not directly release NO are preferably ethyl nitrite or ethyl nitrate.

Ethyl nitrite is available commercially, e.g., diluted in ethanol. Ethyl nitrite (ENO) is a relatively low-molecular-weight colorless organic nitrite with a density of 0.9. ENO is highly volatile and readily decomposes in biologic mediums to produce endogenous mediators of NO bioactivity. Ethyl nitrite forms S-nitrosothiols more readily than does NO, and resists higher-order NO formation.

ENO is administered by inhalation in an amount of 0.1 to 5,000 ppm, preferably 0.1 to 2,000 ppm, more preferably 0.1 to 2,000 ppm, even more preferably 1 to 200 ppm ENO, or 50 to 200 ppm.

ENO₂ is administered in an amount of 1.0 to 2000 ppm, preferably 1 to 200 ppm, and more preferably 50 to 200 ppm. ENO₂ is also administered in gaseous form in a manner similar to ENO. ENO₂ also mimics the effect of NO by formation of S-nitrosothiols. ENO₂ appears to have a lower tendency than NO to generate toxic species on reaction with O₂, and exhibits a lower risk of inducing methemoglobinemia.

Red blood cell nitrosylating agents that do not directly release NO are optionally administered with other NO and/or NO donor compounds discussed above.

In another aspect of this embodiment, the donor and/or organ recipient are treated after the organ has been transplanted. The type of organs, compounds, amounts, and manner in which these compounds are administered are the same as discussed above.

In yet another aspect of this embodiment, the donor and/or organ recipient are treated during the organ transplant procedure. The type of organs, compounds, amounts, and manner in which these compounds are administered are the same as discussed above.

In an even further aspect of this embodiment, the donor and/or organ recipient are treated before, during an organ transplantation procedure, and after the organ has been transplanted. The type of organs, compounds, amounts, and manner in which these compounds are administered are the same as discussed above.

The manner in which the NO, NO donor compound and/or mixtures thereof are delivered to the organ will vary and depend in part on the status of the donor. The donor may be living, brain dead, non-heart beating, or cadaveric.

During a living donor organ procurement procedure (e.g. living donor nephrectomy or partial hepatectomy), the NO, a NO donor compound and/or a mixture thereof is administered to the living donor by inhalation or insufflation. For example, when the NO, a NO donor compound and/or a mixture thereof is ethyl nitrite (ENO), the administration of ethyl nitrite by inhalation is preferably accomplished by a delivery device designed for this purpose. The transplant team removing the organ will adjust the device settings in response to changes in the blood gas status and organ blood flow of the patient.

The NO, NO donor compound or a mixture thereof is administered to a brain dead, non-heart beating, or cadaveric donor by inhalation, ventilation, and/or intra or extra vascular aeration. A brain dead donor is typically a donor wherein lung and/or cardiac function is initially present whereas a non-heart beating, or cadaveric donor is typically a donor, wherein lung and/or cardiac function is compromised or non-existent.

Intravascular aeration refers to the technique where a catheter is placed in a large vein. Such a catheter typically contains a cylindrical bundle of microporous hollow fiber membranes woven into a mat at the end. The catheter is placed within the central venous blood stream in the primary vein that returns blood to the heart (e.g. the inferior vena cava). The device is initially inserted percutaneously or via open venotomy into a large peripheral vessel (e.g. the femoral vein) and then threaded into the inferior vena cava where the hollow fibers encounter all the blood flowing back to the heart. A Respiratory System is activated and ENO along with oxygen (O₂) flows from a console outside the patient, through the catheter and through the hollow fibers. The fiber membranes are permeable to gases. As a result, ENO can nitrosylate the blood components to increase NO bioactivity and O₂ diffuses into the blood stream from the fibers, while carbon dioxide (CO₂) diffuses out of the blood stream into the fibers. Excess ENO, O₂ and the “expired” CO₂ are transported back through the catheter to the external console.

Extravascular aeration refers to using a device such as an extracorporeal membrane oxygenation (ECMO) machine used on a donor or body part or a subject in need of organ transplantation or placing a donor or subject in need of organ preservation on cardio pulmonary bypass (CPB). An ECMO is an extracorporeal technique of providing both cardiac and respiratory support to patients whose heart and lungs are so severely diseased or damaged that they can no longer serve their function (e.g., see US Pat. No. 7,473,239). For both ECMO and CPB the same concepts of intravascular aeration apply (i.e. administration of ENO and O₂ into the circulating blood and removal of CO₂).

In yet another facet of this embodiment, an ex vivo solution is provided that facilitates the preservation of an ex vivo organ or body part requiring a continual supply of oxygen and nutrients. Because most transplanted organs are from deceased donors, the organ must be stored after its removal from the donor until it can be transplanted into a suitable recipient. The donor and recipient are often in different locations, and time is needed to transport the donor organ to the hospital where the recipient is being prepared for transplantation.

The ex vivo solution can be also be used in combination with the administration of NO, NO donor compounds, and/or mixtures thereof as discussed above in procedures such as perfusion. Perfusion is the the act of pouring over or through, especially the passage of a fluid through the vessels of a specific organ. This feature takes into account that blood inactivates NO and uses blood nitrosylation to inhibit and/or overcome the NO-inactivating effects of blood.

The ex vivo solution comprises a red blood cell nitrosylating agent in gaseous form that does not directly release NO as discussed above. Examples of such compounds are ethyl nitrite, ethyl nitrate, or a mixture thereof.

The red blood cell nitrosylating agents in gaseous form that do not directly release NO are incorporated into a variety of solutions, such as continuous pulsatile perfusion solutions and hypothermic storage solutions.

In pulsatile perfusion, the organ is subjected to pulsatile flow of a perfusate under hypothermic conditions such that the organ membranes receive sufficient oxygenation. Typically, the perfusate contains various ions, sugars, and starches along with insulin and dexamethasone.

With hypothermic storage, organs are removed from a brain-dead, non-heart beating or cadaver donor and rapidly cooled. Rapid cooling is achieved by external cooling and by perfusion with a preservative solution to lower the internal temperature of the organ. The organ is then immersed and stored in the preservative solution at temperatures of about 0°-4° C.

These methods in combination with the administration of a preservative solution comprising a red blood cell nitrosylating agent in gaseous form that does not directly release NO allow for longer ex vivo storage time of the organ. Longer storage times provide additional time for histocapability testing of the donor and recipient, organ viability testing and provides additional time to make preoperative decisions and preparations.

These preservative solutions contain a variety of compounds which act as osmotic agents to prevent cell swelling and thereby protect the organs from swelling associated with cellular necrosis during storage. The degree of necrosis occurring in a stored organ can be observed by using conventional light microscopy with fixed tissue samples.

These solutions include but are not limited to Euro-Collins solution, Ross-Marshall citrate solution, Bretschneider Histidine Tryptophan Ketoglutarate solution, University of Wisconsin solution, Celsior solution, and Kyoto ET solution. Two examples of preservative flush solutions are the Collins (G. M. Collins, The Lancet, 1969, 1219-1222, the entire contents of which are hereby incorporated by reference) and the Euro-Collins (J. P. Squifflet et al, Transplant. Proc., 1981, 13:693-696, the entire contents of which are hereby incorporated by reference) solutions. These solutions resemble intracellular fluid and contain glucose as an osmotic agent.

In addition to glucose, high osmolality preservative solutions have been prepared using raffinose and lactobionate such as the University of Washington (UW) preservative solution (R. J. Ploeg et al, Transplant. Proc., 1988, 20 (suppl 1) 1:935-938), mannitol in the Sacks solution (S. A. Sacks, The Lancet, 1973, 1:1024-1028), sucrose in the phosphate buffered sucrose (PBS) preservative solution (F. T. Lam et al, Transplantation, 1989, 47:767-771) and the histidine buffered HTK solution of Bretschneider (N. M. Kallerhoff et al, Transplantation, 1985, 39:485-489). Hypertonic citrate preservative solutions are also known (e.g., H. Ross et al, Transplantation, 1976, 21:498-501). The entire content of each publication is hereby incorporated by reference.

Preservative solutions are also known which contain synthetic hydroxyethyl starch (HES) as an osmotic colloid. The HES has an average molecular weight of about 150,000 to about 350,000 daltons and a degree of substitution of from about 0.4 to about 0.7 (See U.S. Pat. No. 4,879,283 and U.S. Pat. No. 4,798,824). U.S. Pat. No. 5,082,831 discloses a total body washout perfusion solution containing high molecular weight (500,000 daltons) HES. The HES washout solution produces substantially less edema than conventional washout solutions containing DEXTRAN 40 as a colloid. Solutions containing DEXTRAN 40 produce edema, particularly in the pancreas and lungs. The entire contents of each patent hereby incorporated by reference

Preservative solutions are also known for preserving corneas for transplantation. Corneal preservative solutions are designed to prevent endothelial cell damage. Corneal preservative solutions containing glucose or dextran are known (e.g., H. E. Kaufman et al, Arch. Ophthalmol., 1991, 109:864-868; B. E. McCarey and H. E. Kaufman, 1974, Invest. Ophthalmol., 1974, 13:859; B. E. McCarey and H. E. Kaufman, Invest. Ophthamol., 1974, 13:165, the entire contents of each publication are hereby incorporated by reference). The corneal preservative solutions known as OPTISOL, DEXSOL and MK contain DEXTRAN 40 (average molecular weight=40,000 daltons) as an osmotic agent at a concentration of 1-5 wt %.

One feature of this embodiment is a composition comprising (i) at least one red blood cell nitrosylating agent in the gaseous form that does not directly release NO, and (ii) a preservative solution as discussed above. Alternatively, the composition comprises (i) at least one red blood cell nitrosylating agent in the gaseous form that does not directly release NO, and (ii) at least one ingredient of a preservative solution as discussed above.

The red blood cell nitrosylating agent in the gaseous form that does not directly release NO are in accordance with those discussed above and are preferably ethyl nitrite or ethyl nitrate or amyl nitrite.

As to the ingredients of the preservative solution, the composition preferably contains at least one, at least two, at least three, or at least four ingredients selected from the group consisting of blood, blood components, ions, sugars, starches, potassium, sodium, magnesium, lactobionate, phosphate 25, sulphate, raffinose, adenosine, allopurinol, glucose, citrate, mannitol, histidine, glutathione, insulin, dexamethasone, hydroxyethyl starch, bactrim, tryptophan, alpha-ketoglutaric acid, and mixtures thereof.

For example, an ex vivo solution in accordance with the invention comprises (i) red blood cell nitrosylating agents in gaseous form that does not directly release NO and (ii) potassium, sodium, magnesium, lactobionate, phosphate 25, sulphate, raffinose, adenosine, allopurinol, glucose, citrate, mannitol, histidine, glutathione, insulin, dexamethasone, hydroxyethyl starch, bactrim, tryptophan and/or alpha-ketoglutaric acid. The solution has an osmolality of 250-450 mmol/kg and pH of 6.6-7.8 at room temperature.

The red blood cell nitrosylating agent in the gaseous form that does not directly release NO is incorporated into a preservative solution with an aeration device. For example, a small hollow-fiber membrane oxygenator kit as used in a cardio bypass circuit unit can be used to limit bubble formation in the circulating preservative solution containing the at least one red blood cell nitrosylating agent in the gaseous form that does not directly release NO.

The ex vivo solution is used to facilitate the preservation of organs such as a kidney, skin, muscle, heart, lung, liver, cornea, pancreas, islets of Langerhans, intestine, heart valve, stem cells, bone marrow, blood, neural tissue, and composite tissues (e.g. facial allotransplantation).

We turn now to the second embodiment herein.

Altitude sickness, also known as acute mountain sickness (AMS), altitude illness, or hypobaropathy, is a pathological effect resulting from impaired lung function and/or high altitude on humans and animals (e.g. cats, dogs, mules, sheep and the like). For example, altitude sickness can result from acute exposure to low air pressure. It commonly occurs above altitudes of approximately 8,000 feet.

If not treated, altitude sickness can progress to high altitude cerebral edema (HACE) or high altitude pulmonary edema (HAPE). HACE is defined as the onset of ataxia (altered balance or coordination), altered consciousness or both in someone with AMS or HAPE. The classis symptoms of HACE are the usual symptoms of AMS plus confusion, hallucination, diminished levels of consciousness progressing to coma. HAPE is potentially fatal and accounts for most of the deaths from high altitude illness. HAPE is similar to AMS in that the incidence is related to the rate of ascent. The predominant symptom of HAPE is dyspnea or shortness of breath with reduced exercise tolerance or performance. There is often a dry cough with subsequently progresses to a cough that produces frothy bloody sputum. The heart rate and respiratory rate are increased and mild fever is common.

The rate of ascent, altitude attained, amount of physical activity at high altitude, as well as individual susceptibility, and are contributing factors to the onset and severity of high-altitude illness.

Acclimatization is an adaptive process that allows humans and animals to tolerate high altitude. The process of acclimatization begins immediately but requires several days to be notable and sometimes requires weeks to complete. Humans at extreme altitude can require over a month to complete the acclimatization process. The process of acclimatization cannot be rushed, and this explains why individuals (e.g. climbers, soldiers, war-fighters) need to spend days (or even weeks at times) acclimatizing before attempting to climb a high peak.

It has been shown that the inhalation of NO improves arterial oxygenation in high-altitude pulmonary edema. However, NO is a highly reactive and readily diffusible radical. As a result, the administration of NO can generate toxic species on reaction with O2 that induce disorders such as oxidative stress and methemoglobinemia. (See Coggins and Bloch, Arteriosclerosis, Thrombosis, and Vascular Biology. 2007:27:1877). In addition, NO is inactivated by blood and thus systemic activities are limited. A NO donor compound can be administered as a substitute for NO or in combination with NO in an effort to avoid these deleterious effects. The NO donor compounds are preferably red blood cell nitrosylating agents that do not directly release NO.

The inventors of the present application have discovered a method for treating a subject having or at risk of developing high altitude illness, high altitude pulmonary edema and/or acute mountain sickness, comprising administering to the subject in need thereof a therapeutically effective amount of red blood cell nitrosylating agents in the gaseous form that do not directly release NO.

A subject at “risk of developing high altitude illness, high altitude pulmonary edema and/or acute mountain sickness” is a subject that does not yet have high altitude illness, high altitude pulmonary edema and/or acute mountain sickness but is prospectively treated to inhibit the onset of these disorders. This includes treating the subject to facilitate a subject's physiologic adaptation to high altitude environments. For example, a subject may be treated at or near sea level, will be travelling to an area of altitude of 3500 m or higher within a week, 48 hours, or 24 hours.

The type of red blood cell nitrosylating agents that do not directly release NO, amounts, and manner in which these red blood cell nitrosylating agents are administered are the same as discussed above. The of red blood cell nitrosylating agents in gaseous form that does not directly release NO can also be administered via a portable gas delivery unit. In one aspect of this embodiment, the portable gas delivery unit comprises a bottle containing the red blood cell nitrosylating agent in gaseous form that does not directly release NO, a mask or nasal cannula, and/or a regulator.

The red blood cell nitrosylating agents in gaseous form that do not directly release NO are preferably ethyl nitrite, ethyl nitrate, or a mixture thereof. ENO is administered by inhalation in an amount of 0.1 to 2,000 ppm, preferably 0.1 to 1,000 ppm, more preferably 1 to 200 ppm ENO, and even more preferably 50 to 200 ppm.

ENO₂ is administered in an amount of 1.0 to 2000 ppm, preferably 1 to 200 ppm, and more preferably 50 to 200 ppm. ENO₂ is also administered in gaseous form in a manner similar to ENO.

In one facet of this embodiment, a red blood cell nitrosylating agent in gaseous form that does not directly release NO is administered to a subject, wherein the subject is hypoxemic and other drugs cannot change oxygenation. The administration of a red blood cell nitrosylating agent in gaseous form that does not directly release NO (e.g., ENO, ENO₂ or mixtures thereof), protects against the toxicity of high altitude illness by improving tissue oxygenation and metabolism.

The administration of red blood cell nitrosylating agents in gaseous form that do not directly release NO are optionally administered in combination with N-acetyl cysteine in an amount of 200-1000 milligrams P.O. TID (by mouth, three times a day), ascorbic acid, dexamethasone, acetazolamide, a phosphodiesterase inhibitors (e.g., dypiridamol and sildenafil), ibuprofen, or nifedipine.

Acetazolamide helps some people to speed up the acclimatization process when taken before arriving at altitude, and can treat mild cases of altitude sickness. A typical dose of Acetazolamide is 100-500 mg 1-3 times daily starting the day before moving to altitude. Acetazolamide allows one to breathe faster so that the person metabolizes more oxygen, thereby minimizing the symptoms caused by poor oxygenation.

Dexamethasone is a prescription drug that decreases brain and other swelling reversing the effects of AMS. A dosage is typically 1-8 mg, 1-4 times a day a day starting with the ascent. This inhibits some symptoms of altitude illness.

Ibuprofen is effective at relieving altitude headache.

Nifedipine rapidly decreases pulmonary artery pressure and can relieve HAPE.

Additional treatments such as administering oxygen to the patient or placing the patient in a Gamow bag can be practiced in conjunction with the invention. Breathing oxygen reduces the effects of altitude illnesses. Oxygen enrichment can counteract the effects of altitude sickness, or hypoxia. A small amount of supplemental oxygen reduces the equivalent altitude in climate-controlled rooms. For example, a Gamow bag is an inflatable pressure bag that acts as a hyperbaric chamber; it is designed to house a person inside. By inflating the bag with a foot pump, the effective altitude can be decreased as much as 5,000 feet. It is primarily used for treating severe cases of altitude sickness.

Background and working examples for the invention are set forth below.

### EXAMPLE 1

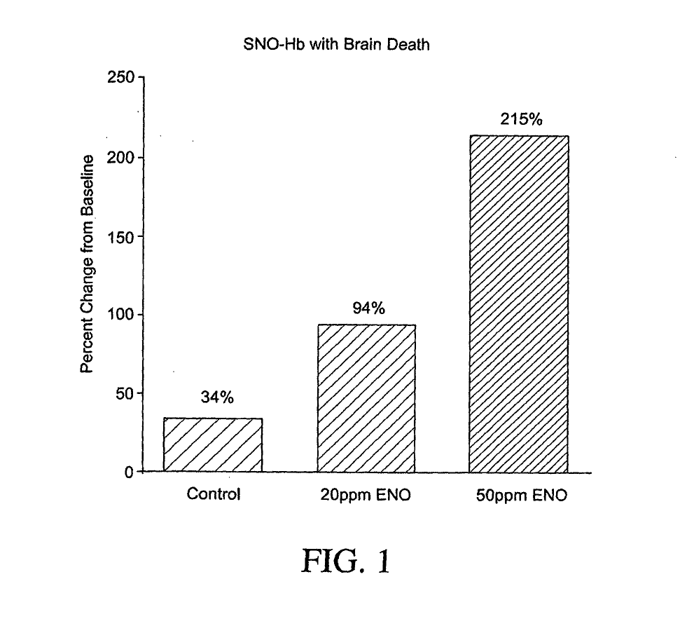

The level of circulating SNO-Hb following brain death in three groups of swine was measured. Animals in the control group (n=10) exhibited declines in circulating SNO levels while animals that were ventilated with 20 ppm (n=14) or 50 ppm ENO (n=14) exhibited an increase in SNO-Hb. An increase in SNO-Hb ties in with the reduction in markers of organ injury, creatinine and AST. FIG. 1 is a bar graph illustrating the results of administering ENO to an organ after brain death. The chart shows the percent change from baseline in red blood cell SNO-Hb concentration 12 hours after brain death.

### EXAMPLE 2

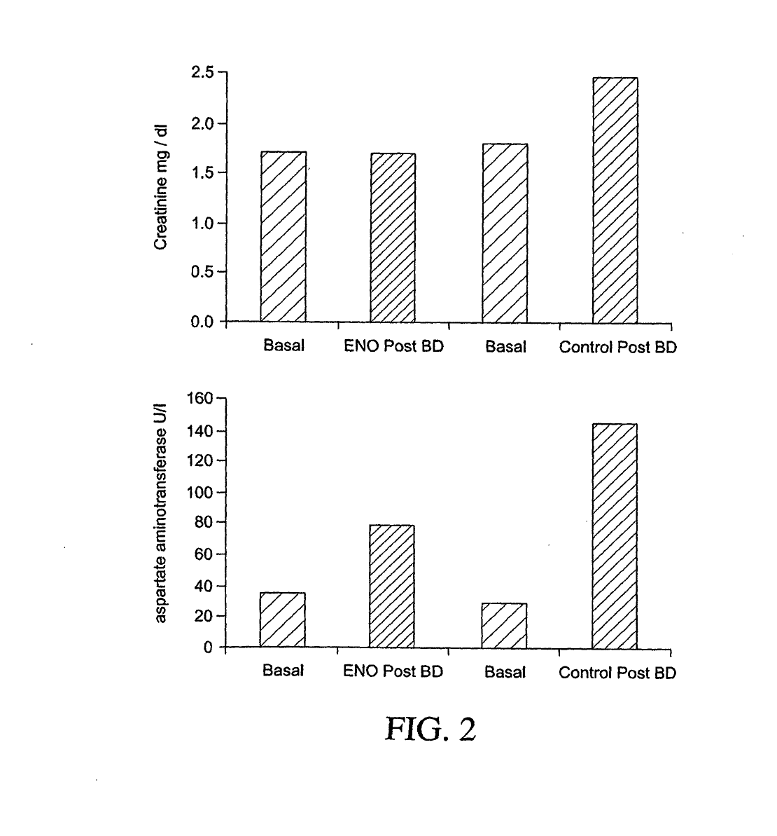

The levels of creatinine and aspartate aminotransferase (AST) were monitored in two groups of swine following brain death. The level of creatinine is indicative of kidney function. The level of AST is indicative of liver function. For each group, a base line blood sample was taken, brain death was induced, and the second sample was taken after 12 h with or without 50 ppm ENO mixed into the ventilation circuit. FIG. 2 shows that creatinine levels went from 1.8 to 2.5 mg/dl in the control no ENO group whereas levels went unchanged with the groups that was administered ENO. AST increased in both groups but the magnitude of the increase with ENO was half that observed in the control group (35 to 79 U/l v. 30 to 145 U/l). The results indicated that administration of ENO preserved kidney function and had a beneficial effect on liver function.

### EXAMPLE 3

A kidney is preserved for organ transplant by perfusing the kidney with a composition containing University of Washington (UW) solution and ENO in the UW solution at 50 ppm. The solution is rinsed off after several hours and the kidney is transplanted in recipient.

### EXAMPLE 4

Skin is preserved for organ transplant by perfusing the tissue with a composition containing UW solution and ENO in the UW solution at 100 ppm. The solution is rinsed off after several hours and the skin is transplanted in recipient.

### EXAMPLE 5

A brain dead donor receives 20 ppm ENO through the ventilation circuit. The facial bloc is harvested en-mass, washed with heparin-saline, and placed in a UW solution bubbled with 50 ppm ENO until the recipients facial area is de-bulked then the procured facial flap is attached.

### EXAMPLE 6

A heart is preserved for organ transplant by perfusing the organ with a composition containing UW solution and ENO in the solution at 50 ppm. The solution is rinsed off after several hours and the heart is transplanted in recipient.

### EXAMPLE 7

A cornea is preserved for transplant by perfusing the cornea with a composition containing preservative solution (i.e., OPTISOL) and ENO at a concentration of 100 ppm. The solution is rinsed off after several hours and the cornea is transplanted in recipient.

### EXAMPLE 8

A living kidney donor is administered ENO via inhalation during an open nephrectomy or as part of the insufflation gas during a laparoscopic donor nephrectomy. ENO is provided in pressurized cylinders for delivery through the ventilation or insufflation devices. The amount of ENO delivered can be titrated based on blood gas or organ blood flow changes.

A kidney is removed from the organ donor and successfully transplanted into recipient.

### EXAMPLE 9

A catheter is placed in a large vein of a brain dead patient. The catheter contains a cylindrical bundle of microporous hollow fiber membranes woven into a mat at the end. The catheter is placed within the central venous blood stream in the primary vein that returns blood to the heart. The device is initially inserted percutaneously or via open venotomy into a large peripheral vessel (e.g. the femoral vein) and then threaded into the inferior vena cava where the hollow fibers encounter all the blood flowing back to the heart. A respiratory system is activated and oxygen with 50 ppm ENO flows from a console outside the patient, through the catheter and through the hollow fibers. The fiber membranes are permeable to gases. As a result, oxygen and ENO diffuses into the blood stream from the fibers, while carbon dioxide (CO₂) diffuses out of the blood stream into the fibers. Excess O₂ and CO₂ are removed back through the catheter to the external console. The liver is removed and successfully transplanted into a recipient.

### EXAMPLE 10

Patient on respiratory is pronounced dead. Creatine phosphokinase (CPK) leak is indicative of cardiac injury. Patient is started on ENO 20 ppm and further CPK leak is prevented. 24 hours later the heart is harvested and successfully transplanted.

### EXAMPLE 11

Patient dies. ENO is begun at 20 ppm and renal function does not decline. Kidney function is preserved over durations usually associated with decline in function. Kidney is successfully transplanted and neither early nor late rejection is observed.

### EXAMPLE 12

A physiological response of sheep to a simulated altitude of −4,500 meters with or without ENO is measured. Systemic vascular resistance (SVR; dynes*sec-1*cm5), pulmonary arterial pressure (PAP; mm Hg), and cardiac output were continuously recorded and are presented as 1 min averages. Pulmonary vascular resistance (PVR; Wood Units) data for the hypoxia alone (shaded) and 50 ppm ENO (open) animals were derived by conducting pulmonary wedges at discrete intervals. Data are group means were taken from 10 sheep per cohort.

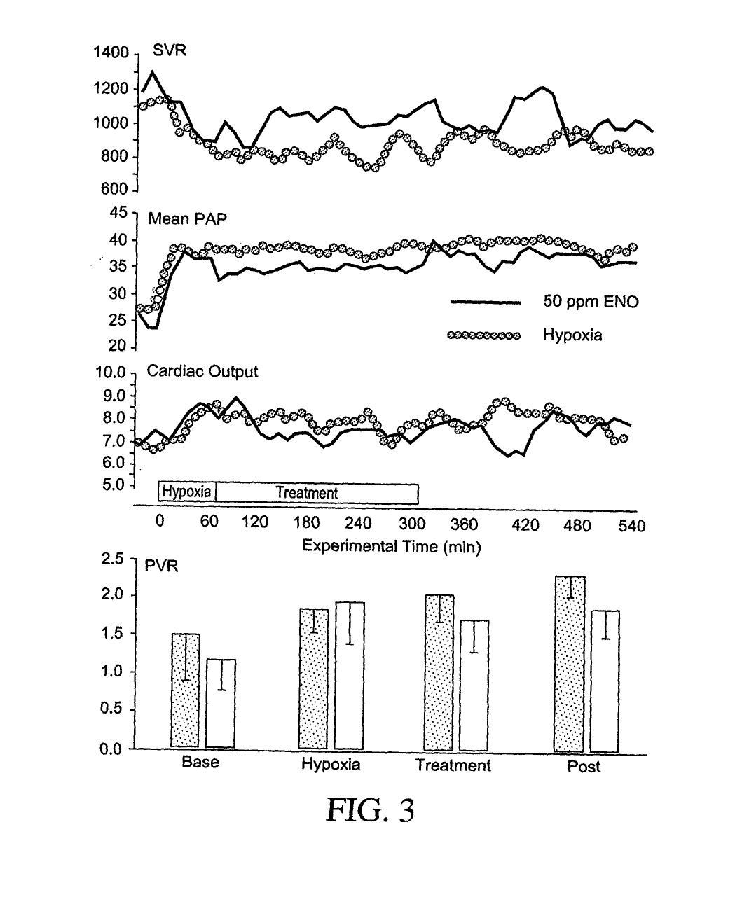

It was found that inhalation of 50 ppm ENO significantly improved physiologic status with respect to restoring SVR and reducing hypoxial high-altitude-induced increases in PAP, cardiac output, and PVR. Results are shown in FIG. 3.

### EXAMPLE 13

A 50-year-old male climber is diagnosed as having altitude sickness. Climber is administered dexamethasone and ENO. Symptoms of altitude mountain sickness abate. Equivalent amounts of ENO₂ provide similar results.

### EXAMPLE 14

A 35-year-old male climber is diagnosed as having altitude mountain sickness. Climber is administered N-acetylcysteine at 300 mg po. TID and ENO at 100 ppm. Symptoms of AMS abate. Symptoms of altitude mountain sickness abate. Equivalent amounts of ENO₂ provide similar results.

### EXAMPLE 15

A 45-year-old female climber is diagnosed with high altitude pulmonary edema. Climber is administered N-acetylcysteine at 1.0 gm IV Q6 and ENO at 100 ppm. Symptoms abate climber is successfully moved to lower altitude. Equivalent amounts of ENO₂ provide similar results.

### EXAMPLE 16

A 25-year-old female preparing for a climb at high altitude is administered ENO and Acetazolamide at a dose of 250 mg twice daily for three days before moving to altitude. Patient exhibits no sign of altitude sickness once at altitude. Equivalent amounts of ENO₂ provide similar results.

### EXAMPLE 17

A 39-year-old male climber is diagnosed as having altitude mountain sickness. Climber is administered N-acetylcysteine at 450 mg po. TID and ENO at 200 ppm. Symptoms of altitude mountain sickness abate. Symptoms of altitude mountain sickness abate and climber is moved to lower altitude. Equivalent amounts of ENO₂ provide similar results.

### EXAMPLE 18

A 42-year-old male climber is diagnosed with high altitude cerebral edema. Climber is administered N-acetylcysteine at 1.5 gm IV Q6 (every six hours) and ENO at 75 ppm. Symptoms abate and climber is moved to lower altitude. Equivalent amounts of ENO₂ provide similar results.

### EXAMPLE 19

A company of United States Special Forces soldiers are rapidly deployed to 10,000 ft elevation. Each soldier is provided with an ENO delivery device that delivers a metered amount of ENO upon inspiration. ENO is continually available during the three day mission. No individuals experiences Acute Mountain Sickness; the mission is successful and the company is returned to sea level. Equivalent amounts of ENO₂ provide similar results.

### EXAMPLE 20

A pet owner presents at a state veterinary college with her cat that was recently struck by car. The animal exhibits no central nervous system activity but is still breathing and has a heart beat. The owner is informed that her cat is brain dead and then provided details about a feline kidney donation program—the owner agrees to have her pet's kidneys transplanted. The animal is intubated and ventilated with oxygen augmented with 50 ppm ENO to preserve organ physiologic status while potential recipients are identified. Two clients of the vet school who own cats with end-stage renal disease are contacted and are grateful for the opportunity to have their pets receive a healthy kidney. 24 hours after presentation the kidneys are procured and transplanted into the two other cats; administration of ENO maintained organ function of the brain dead cat so the grafts function well after transplantation.

### EXAMPLE 21

A barren of mules is purchased by the Department of Defense from a breeder in Tennessee. The mules are flown to the United States Air Force base at Jalalabad, Afghanistan (elevation 1,800 ft). Once on the ground, each animal receives daily inhalational therapy with ENO (100 ppm) and their water is supplemented with NAC. Two days later, the mules are loaded up for a 14 day mission into the Tora Bora mountain range. ENO and NAC are administered as needed to ensure optimal exercise performance at altitude as the mules transport supplies and equipment to a 10,000 ft elevation base camp for the special forces team described in Example 19.

### EXAMPLE 22

A 72 year old grandmother with mild COPD living in San Francisco wants to visit her grandchildren in New York state. On her last commercial flight she experienced continual shortness of breath due to the in-flight reduction in cabin pressure (typically down to 0.8 atmospheres, the equivalent of 8,000-12,000 feet altitude), which was very stressful. For this flight she obtains an individual-use ENO inhaler (set at 20 ppm) from which she takes a puff every 15-20 min for symptomatic relief of dyspnea; the 5 hour flight is uneventful and she arrives in New York breathing normally.

### EXAMPLE 23

A 35 year-old male with obesity-hypoventilation syndrome plans to spend one month at a weight-reduction center in the Colorado Rockies (elevation 9,740 feet). To ensure adequate oxygenation during the start of his diet, he takes 600 mg NAC prior to driving to the resort—the regimen is supplemented by thrice daily use of an ENO inhaler delivering 80 ppm per dose. The combination therapy ensures that his breathing disorder is not exacerbated by the change in altitude and he can focus on completing the diet and exercise regimen.

### Variations

The foregoing description of the invention has been presented describing certain operable and preferred embodiments. It is not intended that the invention should be so limited since variations and modifications thereof will be obvious to those skilled in the art, all of which are within the spirit and scope of the invention.

1. A method for preserving a body part or subject requiring a continual supply of oxygen, comprising administering to said body part or subject a compound selected from the group consisting of NO and/or a NO donor in an amount sufficient to facilitate a supply of oxygen to the body part or subject, and wherein the body part or subject is being readied for transplantation or treated for high altitude pulmonary edema and/or acute mountain sickness.

2. A method for preserving a body part from a donor or subject, comprising administering to said donor or subject NO, a NO donor compound, or mixtures thereof in an amount sufficient to facilitate a supply of oxygen, maintain cellular metabolic activity and maintain function of said body part, wherein the donor or subject is living, brain dead, non-heart beating, or cadaveric.

3. The method according to claim 2, wherein the NO donor compound is a red blood cell nitrosylating agent in gaseous form that does not directly release NO.

4. The method according to claim 3, wherein the red blood cell nitrosylating agent is administered by inhalation, ventilation, or insufflation.

5. The method according to claim 3, wherein the red blood cell nitrosylating agent is administered in a gas at respiration rates, tidal volumes, or insufflation rates and pressures consistent with standard clinical practice.

6. The method according to claim 2, wherein the donor is brain dead, non-heart beating, or cadaveric.

7. The method according to claim 3, wherein the donor is brain dead, non-heart beating, or cadaveric.

8. The method according to claim 3, wherein the compound is administered by an intravascular catheter.

9. The method according to claim 3, wherein the compound is administered by extra-corporeal membrane oxygenator.

10. The method according to claim 3, wherein the compound is administered by placing the deceased donor on cardiopulmonary bypass.

11. The method according to claim 3, wherein the red blood cell nitrosylating agent is ethyl nitrite.

12. The method according to claim 11, wherein the compound is in a concentration of 0.1 to 5,000 ppm, preferably 0.1 to 2,000 ppm, more preferably 0.1 to 2,000 ppm, even more preferably 1 to 200 ppm, or 50 to 200 ppm ethyl nitrite.

13. The method according to claim 3, wherein the body part is selected from the group consisting of kidney, skin, muscle, heart, lung, liver, cornea, pancreas, islets of Langerhans, intestine, stem cells, bone marrow, blood, neural tissue, and composite tissues (e.g. facial allotransplantation).

14. The method according to claim 6, wherein the body part is selected from the group consisting of kidney, skin, muscle, heart, lung, liver, cornea, pancreas, islets of Langerhans, intestine, stem cells, bone marrow, blood, neural tissue, and composite tissues (e.g. facial allotransplantation).

15.-46. (canceled)

47. A composition comprising (i) at least one red blood cell nitrosylating agent in the gaseous form that does not directly release NO, and (ii) a preservative solution comprising an ingredient selected from the groups consisting of ions, sugars, starches, insulin, dexamethasone, blood, blood components, and mixtures thereof.

48. (canceled)

49. The method according to claim 2 wherein said administering to said donor is a nitric oxide donor compound.

50. The method according to claim 49, wherein said nitric oxide donor compound is a red blood cell nitrosylating agent in gaseous form that does not directly release NO.

51. The method according to claim 50, wherein said nitric oxide donor compound is selected from the group consisting of ethyl nitrite, ethyl nitrate, amylnitrite, S-nitrocysteine, S-nitrisoglutathione and mixtures thereof.

52. The method according to claim 51 wherein said nitric oxide donor compound is ethyl nitrite.

53. The method according to claim 50, wherein said nitric oxide donor compound is ethyl nitrate.
